SEQUENCE LISTING (7 sequences)

SEQ ID NO: 1 - DNA, 5425 bp, Artificial Sequence
  DNMT1 nucleotide sequence
ggctccgttccatccttctgcacagggtatcgcctctctccgtttggtacatcccctcctcccccacgcccggactggggtggtagacgccgcctccgctcatcgcccctccccatcggtttccgcgcgaaaagccggggcgcctgcgctgccgccgccgcgtctgctgaagcctccgagatgccggcgcgtaccgccccagcccgggtgcccacactggccgtcccggccatctcgctgcccgacgatgtccgcaggcggctcaaagatttggaaagagacagcttaacagaaaaggaatgtgtgaaggagaaattgaatctcttgcacgaatttctgcaaacagaaataaagaatcagttatgtgacttggaaaccaaattacgtaaagaagaattatccgaggagggctacctggctaaagtcaaatcccttttaaataaagatttgtccttggagaacggtgctcatgcttacaaccgggaagtgaatggacgtctagaaaacgggaaccaagcaagaagtgaagcccgtagagtgggaatggcagatgccaacagcccccccaaacccctttccaaacctcgcacgcccaggaggagcaagtccgatggagaggctaagcgttcaagagaccctcctgcctcagcctcccaagtaactgggattagagctgaaccttcacctagccccaggattacaaggaaaagcaccaggcaaaccaccatcacatctcattttgcaaagggccctgccaaacggaaacctcaggaagagtctgaaagagccaaatcggatgagtccatcaaggaagaagacaaagaccaggatgagaagagacgtagagttacatccagagaacgagttgctagaccgcttcctgcagaagaacctgaaagagcaaaatcaggaacgcgcactgaaaaggaagaagaaagagatgaaaaagaagaaaagagactccgaagtcaaaccaaagaaccaacacccaaacagaaactgaaggaggagccggacagagaagccagggcaggcgtgcaggctgacgaggacgaagatggagacgagaaagatgagaagaagcacagaagtcaacccaaagatctagctgccaaacggaggcccgaagaaaaagaacctgaaaaagtaaatccacagatttctgatgaaaaagacgaggatgaaaaggaggagaagagacgcaaaacgacccccaaagaaccaacggagaaaaaaatggctcgcgccaaaacagtcatgaactccaagacccaccctcccaagtgcattcagtgcgggcagtacctggacgaccctgacctcaaatatgggcagcacccaccagacgcggtggatgagccacagatgctgacaaatgagaagctgtccatctttgatgccaacgagtctggctttgagagttatgaggcgcttccccagcacaaactgacctgcttcagtgtgtactgtaagcacggtcacctgtgtcccatcgacaccggcctcatcgagaagaatatcgaactcttcttttctggttcagcaaaaccaatctatgatgatgacccatctcttgaaggtggtgttaatggcaaaaatcttggccccataaatgaatggtggatcactggctttgatggaggtgaaaaggccctcatcggcttcagcacctcatttgccgaatacattctgatggatcccagtcccgagtatgcgcccatatttgggctgatgcaggagaagatctacatcagcaagattgtggtggagttcctgcagagcaattccgactcgacctatgaggacctgatcaacaagatcgagaccacggttcctccttctggcctcaacttgaaccgcttcacagaggactccctcctgcgacacgcgcagtttgtggtggagcaggtggagagttatgacgaggccggggacagtgatgagcagcccatcttcctgacaccctgcatgcgggacctgatcaagctggctggggtcacgctgggacagaggcgagcccaggcgaggcggcagaccatcaggcattctaccagggagaaggacaggggacccacgaaagccaccaccaccaagctggtctaccagatcttcgatactttcttcgcagagcaaattgaaaaggatgacagagaagacaaggagaacgcctttaagcgccggcgatgtggcgtctgtgaggtgtgtcagcagcctgagtgtgggaaatgtaaagcctgcaaggacatggttaaatttggtggcagtggacggagcaagcaggcttgccaagagcggaggtgtcccaatatggccatgaaggaggcagatgacgatgaggaagtcgatgataacatcccagagatgccgtcacccaaaaaaatgcaccaggggaagaagaagaaacagaacaagaatcgcatctcttgggtcggagaagccgtcaagactgatgggaagaagagttactataagaaggtgtgcattgatgcggaaaccctggaagtgggggactgtgtctctgttattccagatgattcctcaaaaccgctgtatctagcaagggtcacggcgctgtgggaggacagcagcaacgggcagatgtttcacgcccactggttctgcgctgggacagacacagtcctcggggccacgtcggaccctctggagctgttcttggtggatgaatgtgaggacatgcagctttcatatatccacagcaaagtgaaagtcatctacaaagccccctccgaaaactgggccatggagggaggcatggatcccgagtccctgctggagggggacgacgggaagacctacttctaccagctgtggtatgatcaagactacgcgagattcgagtcccctccaaaaacccagccaacagaggacaacaagttcaaattctgtgtgagctgtgcccgtctggctgagatgaggcaaaaagaaatccccagggtcctggagcagctcgaggacctggatagccgggtcctctactactcagccaccaagaacggcatcctgtaccgagttggtgatggtgtgtacctgccccctgaggccttcacgttcaacatcaagctgtccagtcccgtgaaacgcccacggaaggagcccgtggatgaggacctgtacccagagcactaccggaaatactccgactacatcaaaggcagcaacctggatgcccctgagccctaccgaattggccggatcaaagagatcttctgtcccaagaagagcaacggcaggcccaatgagactgacatcaaaatccgggtcaacaagttctacaggcctgagaacacccacaagtccactccagcgagctaccacgcagacatcaacctgctctactggagcgacgaggaggccgtggtggacttcaaggctgtgcagggccgctgcaccgtggagtatggggaggacctgcccgagtgcgtccaggtgtactccatgggcggccccaaccgcttctacttcctcgaggcctataatgcaaagagcaaaagctttgaagatcctcccaaccatgcccgtagccctggaaacaaagggaagggcaagggaaaagggaagggcaagcccaagtcccaagcctgtgagccgagcgagccagagatagagatcaagctgcccaagctgcggaccctggatgtgttttctggctgcggggggttgtcggagggattccaccaagcaggcatctctgacacgctgtgggccatcgagatgtgggaccctgcggcccaggcgttccggctgaacaaccccggctccacagtgttcacagaggactgcaacatcctgctgaagctggtcatggctggggagaccaccaactcccgcggccagcggctgccccagaagggagacgtggagatgctgtgcggcgggccgccctgccagggcttcagcggcatgaaccgcttcaattcgcgcacctactccaagttcaaaaactctctggtggtttccttcctcagctactgcgactactaccggccccggttcttcctcctggagaatgtcaggaactttgtctccttcaagcgctccatggtcctgaagctcaccctccgctgcctggtccgcatgggctatcagtgcaccttcggcgtgctgcaggccggtcagtacggcgtggcccagactaggaggcgggccatcatcctggccgcggcccctggagagaagctccctctgttcccggagccactgcacgtgtttgctccccgggcctgccagctgagcgtggtggtggatgacaagaagtttgtgagcaacataaccaggttgagctcgggtcctttccggaccatcacggtgcgagacacgatgtccgacctgccggaggtgcggaatggagcctcggcactggagatctcctacaacggggagcctcagtcctggttccagaggcagctccggggcgcacagtaccagcccatcctcagggaccacatctgtaaggacatgagtgcattggtggctgcccgcatgcggcacatccccttggccccagggtcagactggcgcgatctgcccaacatcgaggtgcggctctcagacggcaccatggccaggaagctgcggtatacccaccatgacaggaagaacggccgcagcagctctggggccctccgtggggtctgctcctgcgtggaagccggcaaagcctgcgaccccgcagccaggcagttcaacaccctcatcccctggtgcctgccccacaccgggaaccggcacaaccactgggctggcctctatggaaggctcgagtgggacggcttcttcagcacaaccgtcaccaaccccgagcccatgggcaagcagggccgcgtgctccacccagagcagcaccgtgtggtgagcgtgcgggagtgtgcccgctcccagggcttccctgacacctaccggctcttcggcaacatcctggacaagcaccggcaggtgggcaatgccgtgccaccgcccctggccaaagccattggcttggagatcaagctttgtatgttggccaaagcccgagagagtgcctcagctaaaataaaggaggaggaagctgctaaggactagttctgccctcccgtcacccctgtttctggcaccaggaatccccaacatgcactgatgttgtgtttttaacatgtcaatctgtccgttcacatgtgtggtacatggtgtttgtggccttggctgacatgaagctgttgtgtgaggttcgcttatcaactaatgatttagtgatcaaattgtgcagtactttgtgcattctggattttaaaagttttttattatgcattatatcaaatctaccactgtatgagtggaaattaagactttatgtagtttttatatgttgtaatatttcttcaaataaatctctcctataaaccaaaaaaaaaaaaaaaaaaaaaaaaaa

SEQ ID NO: 2 - protein, 1632 aa, Artificial Sequence
  DNMT1 amino acid sequence
MPARTAPARVPTLAVPAISLPDDVRRRLKDLERDSLTEKECVKEKLNLLHEFLQTEIKNQLCDLETKLRKEELSEEGYLAKVKSLLNKDLSLENGAHAYNREVNGRLENGNQARSEARRVGMADANSPPKPLSKPRTPRRSKSDGEAKRSRDPPASASQVTGIRAEPSPSPRITRKSTRQTTITSHFAKGPAKRKPQEESERAKSDESIKEEDKDQDEKRRRVTSRERVARPLPAEEPERAKSGTRTEKEEERDEKEEKRLRSQTKEPTPKQKLKEEPDREARAGVQADEDEDGDEKDEKKHRSQPKDLAAKRRPEEKEPEKVNPQISDEKDEDEKEEKRRKTTPKEPTEKKMARAKTVMNSKTHPPKCIQCGQYLDDPDLKYGQHPPDAVDEPQMLTNEKLSIFDANESGFESYEALPQHKLTCFSVYCKHGHLCPIDTGLIEKNIELFFSGSAKPIYDDDPSLEGGVNGKNLGPINEWWITGFDGGEKALIGFSTSFAEYILMDPSPEYAPIFGLMQEKIYISKIVVEFLQSNSDSTYEDLINKIETTVPPSGLNLNRFTEDSLLRHAQFVVEQVESYDEAGDSDEQPIFLTPCMRDLIKLAGVTLGQRRAQARRQTIRHSTREKDRGPTKATTTKLVYQIFDTFFAEQIEKDDREDKENAFKRRRCGVCEVCQQPECGKCKACKDMVKFGGSGRSKQACQERRCPNMAMKEADDDEEVDDNIPEMPSPKKMHQGKKKKQNKNRISWVGEAVKTDGKKSYYKKVCIDAETLEVGDCVSVIPDDSSKPLYLARVTALWEDSSNGQMFHAHWFCAGTDTVLGATSDPLELFLVDECEDMQLSYIHSKVKVIYKAPSENWAMEGGMDPESLLEGDDGKTYFYQLWYDQDYARFESPPKTQPTEDNKFKFCVSCARLAEMRQKEIPRVLEQLEDLDSRVLYYSATKNGILYRVGDGVYLPPEAFTFNIKLSSPVKRPRKEPVDEDLYPEHYRKYSDYIKGSNLDAPEPYRIGRIKEIFCPKKSNGRPNETDIKIRVNKFYRPENTHKSTPASYHADINLLYWSDEEAVVDFKAVQGRCTVEYGEDLPECVQVYSMGGPNRFYFLEAYNAKSKSFEDPPNHARSPGNKGKGKGKGKGKPKSQACEPSEPEIEIKLPKLRTLDVFSGCGGLSEGFHQAGISDTLWAIEMWDPAAQAFRLNNPGSTVFTEDCNILLKLVMAGETTNSRGQRLPQKGDVEMLCGGPPCQGFSGMNRFNSRTYSKFKNSLVVSFLSYCDYYRPRFFLLENVRNFVSFKRSMVLKLTLRCLVRMGYQCTFGVLQAGQYGVAQTRRRAIILAAAPGEKLPLFPEPLHVFAPRACQLSVVVDDKKFVSNITRLSSGPFRTITVRDTMSDLPEVRNGASALEISYNGEPQSWFQRQLRGAQYQPILRDHICKDMSALVAARMRHIPLAPGSDWRDLPNIEVRLSDGTMARKLRYTHHDRKNGRSSSGALRGVCSCVEAGKACDPAARQFNTLIPWCLPHTGNRHNHWAGLYGRLEWDGFFSTTVTNPEPMGKQGRVLHPEQHRVVSVRECARSQGFPDTYRLFGNILDKHRQVGNAVPPPLAKAIGLEIKLCMLAKARESASAKIKEEEAAKD

SEQ ID NO: 3 - DNA, 3999 bp, Artificial Sequence
  UHRF1 nucleotide sequence
gcgcccgcccccggcacggcctcctgcggccccgcaactcccaaatgccgagttttcgcgggaaaaaaatcagagcagctggcagcgcggcgggcagcgtttgccgagcgggcgctccgggtcgcacgcaagtccgcgcggggtccgggccacgcacgcggtttcatcgccatccccagccgggccacgcgcgcaggcagacaagctgttcgcggcgaccggagagcgccgacaccatgtggatccaggttcggaccatggacgggaggcagacccacacggtggactcgctgtccaggctgaccaaggtggaggagctgaggcggaagatccaggagctgttccacgtggagccaggcctgcagaggctgttctacaggggcaaacagatggaggacggccataccctcttcgactacgaggtccgcctgaatgacaccatccagctcctggtccgccagagcctcgtgctcccccacagcaccaaggagcgggactccgagctctccgacaccgactccggctgctgcctgggccagagtgagtcagacaagtcctccacccacggtgaggcggccgccgagactgacagcaggccagccgatgaggacatgtgggatgagacggaattggggctgtacaaggtcaatgagtacgtcgatgctcgggacacgaacatgggggcgtggtttgaggcgcaggtggtcagggtgacgcggaaggccccctcccgggacgagccctgcagctccacgtccaggccggcgctggaggaggacgtcatttaccacgtgaaatacgacgactacccggagaacggcgtggtccagatgaactccagggacgtccgagcgcgcgcccgcaccatcatcaagtggcaggacctggaggtgggccaggtggtcatgctcaactacaaccccgacaaccccaaggagcggggcttctggtacgacgcggagatctccaggaagcgcgagaccaggacggcgcgggaactctacgccaacgtggtgctgggggatgattctctgaacgactgtcggatcatcttcgtggacgaagtcttcaagattgagcggccgggtgaagggagccccatggttgacaaccccatgagacggaagagcgggccgtcctgcaagcactgcaaggacgacgtgaacagactctgccgggtctgcgcctgccacctgtgcgggggccggcaggaccccgacaagcagctcatgtgcgatgagtgcgacatggccttccacatctactgcctggacccgcccctcagcagtgttcccagcgaggacgagtggtactgccctgagtgccggaatgatgccagcgaggtggtactggcgggagagcggctgagagagagcaagaagaaggcgaagatggcctcggccacatcgtcctcacagcgggactggggcaagggcatggcctgtgtgggccgcaccaaggaatgtaccatcgtcccgtccaaccactacggacccatcccggggatccccgtgggcaccatgtggcggttccgagtccaggtcagcgagtcgggtgtccatcggccccacgtggctggcatacacggccggagcaacgacggagcgtactccctagtcctggcggggggctatgaggatgatgtggaccatgggaattttttcacatacacgggtagtggtggtcgagatctttccggcaacaagaggaccgcggaacagtcttgtgatcagaaactcaccaacaccaacagggcgctggctctcaactgctttgctcccatcaatgaccaagaaggggccgaggccaaggactggcggtcggggaagccggtcagggtggtgcgcaatgtcaagggtggcaagaatagcaagtacgcccccgctgagggcaaccgctacgatggcatctacaaggttgtgaaatactggcccgagaaggggaagtccgggtttctcgtgtggcgctaccttctgcggagggacgatgatgagcctggcccttggacgaaggaggggaaggaccggatcaagaagctggggctgaccatgcagtatccagaaggctacctggaagccctggccaaccgagagcgagagaaggagaacagcaagagggaggaggaggagcagcaggaggggggcttcgcgtcccccaggacgggcaagggcaagtggaagcggaagtcggcaggaggtggcccgagcagggccgggtccccgcgccggacatccaagaaaaccaaggtggagccctacagtctcacggcccagcagagcagcctcatcagagaggacaagagcaacgccaagctgtggaatgaggtcctggcgtcactcaaggaccggccggcgagcggcagcccgttccagttgttcctgagtaaagtggaggagacgttccagtgtatctgctgtcaggagctggtgttccggcccatcacgaccgtgtgccagcacaacgtgtgcaaggactgcctggacagatcctttcgggcacaggtgttcagctgccctgcctgccgctacgacctgggccgcagctatgccatgcaggtgaaccagcctctgcagaccgtcctcaaccagctcttccccggctacggcaatggccggtgatctccaagcacttctcgacaggcgttttgctgaaaacgtgtcggagggctcgttcatcggcactgattttgttcttagtgggcttaacttaaacaggtagtgtttcctccgttccctaaaaaggtttgtcttcctttttttttttatttttatttttcaaatctatacattttcaggaatttatgtattctggctaaaagttggacttctcagtattgtgtttagttctttgaaaacataaaagcctgcaatttctcgacaaaacaacacaagattttttaaagatggaatcagaaactacgtggtgtggaggctgttgatgtttctggtgtcaagttctcagaagttgctgccaccaactctttaagaaggcgacaggatcagtccttctctcgggttctggcccccaaggtcagagcaagcatcttcctgacagcattttgtcatctaaagtccagtgacatggttccccgtggtggcccgtggcagcccgtggcatggcgtggctcagctgtctgttgaagttgttgcaaggaaaagaggaaacatctcgggcctagttcaaacctttgcctcaaagccatcccccaccagactgcttagcgtctgagatccgcgtgaaaagtcctctgcccacgagagcagggagttggggccacgcagaaatggcctcaaggggactctgctccacgtggggccaggcgtgtgactgacgctgtccgacgaaggcggccacggacggacgccagcacacgaagtcacgtgcaagtgcctttgattcgttccttctttctaaagacgacagtctttgttgttagcactgaattattgaaaatgtcaaccagattctagaaactgcggtcatccagttcttcctgacaccggatgggtgcttgggaaccgtttgagccttatagatcatttacattcaatttttttaactcagcaagtgagaacttacaagagggtttttttaaaatttttttttctcttaatgaacacattttctaaatgaattttttttgtagttactgtatatgtaccaagaaagatataacgttagggtttggttgtttttgtttttgtattttttttcttttgaaagggtttgttaatttttctaattttaccaaagtttgcagcctatacctcaataaaacagggatattttaaatcacatacctgcagacaaactggagcaatgttatttttaaagggtttttttcacctccttattcttagattattaatgtattagggaagaatgagacaattttgtgtaggctttttctaaagtccagtactttgtccagattttagattctcagaataaatgtttttcacagatagaaaaaaaaaaaaaaaaaaaaaaaaaaaaaaa

SEQ ID NO: 4 - protein, 793 aa, Artificial Sequence
  UHRF1 amino acid sequence
MWIQVRTMDGRQTHTVDSLSRLTKVEELRRKIQELFHVEPGLQRLFYRGKQMEDGHTLFDYEVRLNDTIQLLVRQSLVLPHSTKERDSELSDTDSGCCLGQSESDKSSTHGEAAAETDSRPADEDMWDETELGLYKVNEYVDARDTNMGAWFEAQVVRVTRKAPSRDEPCSSTSRPALEEDVIYHVKYDDYPENGVVQMNSRDVRARARTIIKWQDLEVGQVVMLNYNPDNPKERGFWYDAEISRKRETRTARELYANVVLGDDSLNDCRIIFVDEVFKIERPGEGSPMVDNPMRRKSGPSCKHCKDDVNRLCRVCACHLCGGRQDPDKQLMCDECDMAFHIYCLDPPLSSVPSEDEWYCPECRNDASEVVLAGERLRESKKKAKMASATSSSQRDWGKGMACVGRTKECTIVPSNHYGPIPGIPVGTMWRFRVQVSESGVHRPHVAGIHGRSNDGAYSLVLAGGYEDDVDHGNFFTYTGSGGRDLSGNKRTAEQSCDQKLTNTNRALALNCFAPINDQEGAEAKDWRSGKPVRVVRNVKGGKNSKYAPAEGNRYDGIYKVVKYWPEKGKSGFLVWRYLLRRDDDEPGPWTKEGKDRIKKLGLTMQYPEGYLEALANREREKENSKREEEEQQEGGFASPRTGKGKWKRKSAGGGPSRAGSPRRTSKKTKVEPYSLTAQQSSLIREDKSNAKLWNEVLASLKDRPASGSPFQLFLSKVEETFQCICCQELVFRPITTVCQHNVCKDCLDRSFRAQVFSCPACRYDLGRSYAMQVNQPLQTVLNQLFPGYGNGR

SEQ ID NO: 5 - DNA, 15 bp, Artificial Sequence
  M13 forward primer
gtaaaacgacggcca

SEQ ID NO: 6 - DNA, 17 bp, Artificial Sequence
  M13 reverse primer
caggaaacagctatgac

SEQ ID NO: 7 - protein, 7 aa, Artificial Sequence
  Chemically synthesized peptide
GENGWIZ

(19) United States
(12) Patent Application Publication
(10) Pub. No.: US 2016/0130643 A1
(43) Pub. Date: May 12, 2016

(54) ACCURATE IN VITRO COPYING OF DNA METHYLATION

(71) Applicants: Ite A. Laird-Offringa, South Pasadena, CA (US); Jinkeng Asong, Hanahan, SC (US); Mihaela Campan, Los Angeles, CA (US); Po-Han Chen, Temple City, CA (US); Crystal N. Marconett, Sun Valley, CA (US); Ian Stuart Haworth, Santa Monica, CA (US)

(72) Inventors: Ite A. Laird-Offringa, South Pasadena, CA (US); Jinkeng Asong, Hanahan, SC (US); Mihaela Campan, Los Angeles, CA (US); Po-Han Chen, Temple City, CA (US); Crystal N. Marconett, Sun Valley, CA (US); Ian Stuart Haworth, Santa Monica, CA (US)

(73) Assignee: University of Southern California, Los Angeles, CA (US)

(21) Appl. No.: 14/929,234

(22) Filed: Oct. 30, 2015

Related U.S. Application Data
(63) Continuation of application No. PCT/US2014/035967, filed on Apr. 29, 2014.
(60) Provisional application No. 61/817,840, filed on Apr. 30, 2013.
(60) Provisional application No. 61/881,849, filed on Sep. 24, 2013.

(51) Int. Cl. C12Q 1/68

(52) U.S. Cl. CPC C12Q 1/6848; C12Q 1/686

(57) ABSTRACT

A method of copying a methylated nucleic acid molecule is provided. The method includes copying a nucleic acid molecule into a plurality of nucleic acid molecules; and contacting the plurality of nucleic acid molecules with a DNA methyltransferase enzyme and an E3 ubiquitin ligase. The method results in the copying of the methylated nucleic acid molecule.

### CROSS REFERENCE TO RELATED APPLICATIONS

This application claims the benefit of priority under §119(e) of U.S. Ser. No. 61/881,849, filed Sep. 24, 2013 and U.S. Ser. No. 61/817,840 filed Apr. 30, 2013. The disclosure of the prior application is considered part of and is incorporated by reference in its entirety in the disclosure of this application.

### FIELD OF THE INVENTION

The invention pertains to the field of DNA methylation assays and amplification of methylated DNA.

### BACKGROUND OF THE INVENTION

DNA methylation is a biochemical process involving the addition of a methyl group to the cytosine or adenine DNA nucleotides. DNA methylation stably alters the expression of genes in cells as cells divide and differentiate from embryonic stem cells into specific tissues. The resulting change is normally permanent and unidirectional, preventing a differentiated cell from reverting to a stem cell or converting into another type of tissue. Recent investigations have shown that DNA methylation plays a crucial role in the development of nearly all types of cancer, thus, detection of DNA methylation pattern may be an effective approach to detection of cancer.

For example, aberrant DNA patterns have been observed in a variety of cancers. As the aberrantly methylated DNA is shed into the blood stream, detection of the presence such differentially methylated DNA elements originating from tumors may form the basis for cancer diagnostics. However, the amount of such DNA in the blood stream is typically very small.

Currently DNA methylation detection methods generally rely on sodium bisulfite treatment before it can be used for any PCR-based analyses that specifically detect DNA methylation. To avoid problems of low DNA and lack of sensitivity, sufficient input DNA must be used. This can be very problematic when limited amounts of samples are available. To increase sensitivity, the PCR reactions are sometimes done with two distinct amplifications (“Nested PCR”). This can increase the chance of contaminations, and does not really solve the issue of low starting amounts of DNA.

To circumvent the need for the harmful bisulfite treatment, there are alternative techniques that specifically allow for enrichment of methylated DNA before analysis, such as by immunoprecipitation with methyl-specific antibodies or capture with methyl-binding proteins. However, these approaches do not provide information on individual methyl-Cs, because they precipitate fragments of DNA that carry methyl groups at one or more undefined positions within each DNA fragment. Another alternative is the use of restriction enzymes that are methylation sensitive or target methylated DNA. However this approach is limited to analyzing methylation that falls in/near the restriction enzyme target sites.

Therefore, there still exists a need for better, more, generally applicable and sensitive method of detecting DNA methylation patterns.

### SUMMARY OF THE INVENTION

In light of the above, it is an object of the present invention to devise a method capable of detecting and amplifying minute amounts of methylated DNA in a sample with high sensitivity and accuracy. It is also an object of the present invention to devise cancer diagnostic tests based on detection of aberrant DNA methylation patterns in a subject. These and other objects of the present invention are satisfied, in part, by the unexpected discovery that DNA methyl-transferase, DNMT1, may be combined with its DNA targeting partner, Ubiquitin-like PHD and RING Finger Domain-Containing protein (UHRF1).

In one exemplary embodiment of the present invention, we have solved this problem by expressing recombinant human DNMT1 and UHRF1 (an accessory protein) in E. coli and using the proteins together to methylate hemi-methylated DNA. As stated above, when used alone DNMT1 is inaccurate and introduces methylation where none was present. We have demonstrated herein that adding UHRF1 to the methylation reaction greatly increases the accuracy of DNMT1, preventing it from methylating unmethylated DNA. This breakthrough opens the path to creating “methylation preserving” PCR reactions, which will allow DNA methylation information to be amplified.

According, a first aspect of the present invention is directed to methods for copying methylated DNAs in vitro without introducing new methylations into the copied DNA. Methods in accordance with this aspect of the invention will generally include the steps of denaturing parent DNA samples into single stranded DNAs; copying the parent DNA strands using primers and polymerase to make daughter strands; and methylating the daughter strands with DNMT1 and UHRF1.

A second aspect of the present invention is directed to a methylation-preserving PCR method. Methods in accordance with this aspect of the invention will generally include the steps of denaturing parent DNA samples into single stranded DNAs; copying the parent DNA strands using primers and polymerase to make daughter strands; methylating the daughter strands with DNMT1 and UHRF1; and repeating the process for a desired number of cycles, wherein in each cycle, the methylated daughter stands are taken as new parent strands in the next cycle.

A third aspect of the present invention is directed to a method of detecting and analyzing DNA methylation patterns in a sample. In some embodiments, methods in accordance with this aspect of the invention will generally include performing a methylation-preserving DNA amplification process as described above, following by interrogating the amplified DNA product for methylation patterns. Interrogation of the amplified DNA product may be performed by any methods commonly known in the art. Such methods typically incorporate a bisulfite treatment reaction followed by some sort of PCR, hybridization, or sequencing.

A fourth aspect of the present invention is directed to a reagent kit for performing a DNA methylation preserving PCR reaction. Kits in accordance with this aspect of the invention will generally include reagents for copying methylated DNAs, wherein said reagents comprises DNMT1 and UHRF1; and instructions encoded on a permanent medium wherein said instructions comprises steps for performing methods according to the first, second, or third aspect of the invention as described above. In some preferred embodiments, the DNMT1 and UHRF1 are recombinant DNMT1 and UHRF1 expressed in E. coli.

A fifth aspect of the present invention is directed to a cancer detection/diagnostic assay based on DNA methylation patterns corresponding to a cancer. Assays in accordance with this aspect of the invention will generally include the steps of obtaining a biological sample from a patient suspected of having a cancer; processing the sample and amplifying methylated DNAs in the sample by performing a methylation-preserving amplification reaction as described above (either according to the first aspect or the second aspect); interrogating the amplified DNAs for methylation patterns; comparing the methylation patterns to reference patterns; and determining a diagnosis based on the comparison, wherein if the methylation patterns in the sample deviates from the patterns in the references, a diagnosis of cancer is determined.

Other aspects and advantages of the invention will be apparent from the following description and the appended claims.

### BRIEF DESCRIPTION OF THE DRAWINGS

FIG. 1 shows a schematics representation of UHRF1 domain.

FIG. 2 shows Sau3A1 digests GATC at (A). unmethylated CpG, (B). hemi-methylated CpG, and (C). fully methylated CpG.

FIG. 3 shows an exemplary Sau3A template.

FIG. 4 shows DNMT1 activity assay for a single CpG. The red ball indicates methylation, the colored bars indicate fragments generated by Sau3AI digestion. Note that the 40-nt fragment is only generated when the top strand is methylated, preventing digestion of the middle site. If DNMT1 lacks de novo methylation, there should be no 40 bp fragment when an unmethylated substrate is provided (left panel).

FIG. 5 shows Un- and hemi-methylated DNA treated with the DNMT1 combined with the UHRF1 or UHRF1⁴¹⁵⁻⁷⁹³ and digested by Sau3AI. Lane2 2 and 6 (“sub”) show DNA probes without DNMT1 and UHRF1 treatment. Lanes 3 and 7 (“D”) show DNA treated with DNMT1 alone; de novo methylation was detected on unmethylated DNA probe (40-nt band, lane 3). Lanes 4 and 8 show DNA treated with DNMT1 combined with UHRF1⁴¹⁵⁻⁷⁹³; this fragment of UHRF1 strongly reduced de novo CpG methylation on the unmethylated DNA probe but did not inhibit methylation of the hemi-methylated DNA probe. Lanes 5 and 9 showed that combined with full-length UHRF1, DNMT1 specifically methylates hemi-methylated DNA but not unmethylated DNA. Sub: DNA substrate, D: DNMT1, Ucter: UHRF1⁴¹⁵⁻⁷⁹³, and U: UHRF1.

FIG. 6 shows multi CpG probe. The DNA probe contains nine CpGs (in bold capitals). Incubation with DNMT1+/−UHRF1 allows assessment of DNMT1 specificity. After treatment with bisulfite reagent, unmethylated cytosines are converted to uracil, but 5-methylcytosine would be not affected. The cytosines are not in CpGs so they will be converted to uracil independent of DNMT1/UHRF1 treatment, which is a good control to verify bisulfite sequencing.

FIG. 7 shows exemplary preliminary results of bisulfite sequencing of 9-CpG target. Note that DNMT1 shows substantial de novo DNA methylation activity in the absence of UHRF1, that is completely inhibited in the presence of UHRF1. The occasional unmethylated CpG in the bottom right panel could be due to incomplete bisulfite conversion, which we have noted on occasion. Repeated experiments have shown a full block of de novo DNA DNMT1 methylation activity in spite of long incubation times with DNMT1

FIG. 8 is an illustration showing the structure of DNMT1.

FIG. 9 is an illustration showing the structure of UHRF1.

FIG. 10 is an illustration of a vector including modified DNMT1 useful in an assay of the invention as shown in Example 2.

FIG. 11 is an illustration of a vector including modified UHFR1 useful in an assay of the invention as shown in Example 2.

FIG. 12 is an illustration of a vector including modified DNMT1 useful in an assay of the invention as shown in Example 2.

FIG. 13 is a flow chart and results of a DNMT1 methylation assay as utilized in Example 2.

FIG. 14 is a series of graphical illustrations showing kinetic activity of constructs of Example 2.

FIG. 15 is the nucleotide sequence for DNMT1 (SEQ ID NO: 1) in one embodiment of the invention.

FIG. 16 is the amino acid sequence for DNMT1 (SEQ ID NO: 2) in one embodiment of the invention.

FIG. 17 is the nucleotide sequence for UHRF1 (SEQ ID NO: 3) in one embodiment of the invention.

FIG. 18 is the amino acid sequence for UHRF1 (SEQ ID NO: 4) in one embodiment of the invention.

### DETAILED DESCRIPTION

It is an object of the present invention to devise a method capable of detecting and amplifying minute amounts of methylated DNA in a sample with high sensitivity and accuracy. It is also an object of the present invention to devise cancer diagnostic tests based on detection of aberrant DNA methylation patterns in a subject. These and other objects of the present invention are satisfied, in part, by the unexpected discovery that DNA methyl-transferase, DNMT1, may be combined with its DNA targeting partner, UHRF1.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

Methylated cytosines in vertebrates largely occur in small inverted repeats, so-called CpG dinucleotides, allowing methylation on the Cs of both strands, diagonally across from each other. This enables DNA methylation information to be copied in vivo during the DNA replication that precedes cell division, because the methylation on the parent strand is used to guide the reintroduction of DNA methylation on the daughter strand. This copying of DNA methylation is carried out by the enzyme DNA methyltransferase 1 (DNMT1), a “maintenance” DNA methyltransferase, at the replication fork, in a complex containing many proteins.

In contrast, when DNA is replicated in vitro, for example by using the polymerase chain reaction (PCR), the polymerase ignores the methylation and copies the cytosine in the unmethylated form. DNA methyltransferase would be needed to methylate the new strand at the appropriate positions (across from methylation on the parent strand), thereby turning hemi-methylated DNA back into fully methylated DNA.

In the past, copying of the methylation in vitro has been attempted by following the DNA polymerase reaction with treatment by DNMT1. However, this copying is inefficient and inaccurate, in that previously unmethylated CpGs can become methylated and many methylated CpGs are not methylated. The copying infidelity leads to gain of abnormal methylation and loss of true methylation (Goyal et al, 2006, Nucleic Acids Res.1182-88, the entire content of which is incorporated herein by reference). This problem makes it impossible to carry out PCR of small amounts of DNA to subsequently analyze the DNA methylation patterns because each cycle of PCR would introduce further inaccuracies so that the correct DNA methylation information would be rapidly lost.“Nucleic acid” and “polynucleotide” are used interchangeably herein to refer to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs). As appreciate by one of skill in the art, the complement of a nucleic acid sequence can readily be determined from the sequence of the other strand. Thus, any particular nucleic acid sequence set forth herein also discloses the complementary strand.

“Polypeptide,” “peptide” and “protein” are used interchangeably herein to refer to a polymer of amino acid residues. The terms apply to naturally occurring amino acid polymers, as well as, amino acid polymers in which one or more amino acid residue is an artificial chemical mimetic of a corresponding naturally occurring amino acid.

“Amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, .gamma.-carboxyglutamate, and O-phosphoserine. “Amino acid analogs” refers to compounds that have the same fundamental chemical structure as a naturally occurring amino acid, i.e., an alpha carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid. Amino acids may be referred to herein by either their commonly known three letter symbols or by the one-letter symbols recommended by the IUPAC-IUB Biochemical Nomenclature Commission.

“Conservatively modified variants” applies to both nucleic acid and amino acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

With respect to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologues, and alleles of the invention.

For example, substitutions may be made wherein an aliphatic amino acid (G, A, I, L, or V) is substituted with another member of the group, or substitution such as the substitution of one polar residue for another, such as arginine for lysine, glutamic for aspartic acid, or glutamine for asparagine. Each of the following eight groups contains other exemplary amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

Macromolecular structures such as polypeptide structures can be described in terms of various levels of organization. For a general discussion of this organization, see, e.g., Alberts et al., Molecular Biology of the Cell (3rd ed., 1994) and Cantor and Schimmel, Biophysical Chemistry Part I. The Conformation of Biological Macromolecules (1980). “Primary structure” refers to the amino acid sequence of a particular peptide. “Secondary structure” refers to locally ordered, three dimensional structures within a polypeptide. “Tertiary structure” refers to the complete three dimensional structure of a polypeptide monomer. Domains are portions of a polypeptide that form a compact unit of the polypeptide and are typically 50 to 350 amino acids long. Typical domains are made up of sections of lesser organization such as stretches of β-sheet and α-helices. “Quaternary structure” refers to the three dimensional structure formed by the noncovalent association of independent tertiary units.

The terms “isolated” or “substantially purified,” when applied to a nucleic acid or protein, denotes that the nucleic acid or protein is essentially free of other cellular components with which it is associated in the natural state. It is preferably in a homogeneous state, although it can be in either a dry or aqueous solution. Purity and homogeneity are typically determined using analytical chemistry techniques such as polyacrylamide gel electrophoresis or high performance liquid chromatography. A protein which is the predominant species present in a preparation is substantially purified.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local alignment algorithm of Smith & Waterman, Adv. Appl. Math. 2:482 (1981), by the global alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)). The Smith & Waterman alignment with the default parameters are often used when comparing sequences as described herein.

Another example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403410 (1990), respectively. BLAST and BLAST 2.0 are used, typically with the default parameters, to determine percent sequence identity for the nucleic acids and proteins of the invention. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of bath strands. For amino acid (protein) sequences, the BLASTP program uses as defaults a wordlength (W) of 3, an expectation (E) of 10, and the BLOSUM62 scoring matrix (see Henikoff& Henikoff(1989) Proc. Natl. Acad. Sci. USA 89:10915)). For the purposes of this invention, the BLAST2.0 algorithm is used with the default parameters.

Conservatively modified variants of antibodies of the present invention have at least 80% sequence similarity, often at least 85% sequence similarity, 90% sequence similarity, or at least 95%, 96%, 97%, 98%, or 99% sequence similarity at the amino acid level, with the protein of interest, such as DNMT1 or UHRF1.

As noted, the term “conservatively modified variants” can be applied to both amino acid and nucleic acid sequences. With respect to particular nucleic acid sequences, conservatively modified variants refer to those nucleic acid sequences which encode identical or essentially identical amino acid sequences, or if the nucleic acid does not encode an amino acid sequence, to essentially identical nucleic acid sequences. Because of the degeneracy of the genetic code, a large number of functionally identical nucleic acids encode any given polypeptide. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid.

In one exemplary embodiment of the present invention, this problem has been solved by expressing recombinant human DNMT1 and UHRF1 (an accessory protein) in E. coli and using the proteins together to methylate hemi-methylated DNA. As stated above, when used alone DNMT1 is inaccurate and introduces methylation where none was present. It is demonstrated herein that adding UHRF1 to the methylation reaction greatly increases the accuracy of DNMT1, preventing it from methylating unmethylated DNA. This breakthrough opens the path to creating “methylation preserving” PCR reactions, which will allow DNA methylation information to be amplified.

According, a first aspect of the present invention is directed to methods for copying methylated DNAs in vitro without introducing new methylations into the copied DNA. Methods in accordance with this aspect of the invention will generally include the steps of denaturing parent DNA samples into single stranded DNAs; copying the parent DNA strands using primers and polymerase to make daughter strands; and methylating the daughter strands with DNMT1 and UHRF1.

As used herein, “DNA methylation” refers to chemical modifications in which a methyl group (i.e.—CH₃) is attached to the 5 position of cytosine. It projects away from the part of cytosine that base-pairs to the other strand. DNA methylation is essential for normal development and is associated with a number of key processes including genomic imprinting, X-chromosome inactivation, suppression of repetitive elements, and carcinogenesis. Between 60% and 90% of all CpGs are methylated in mammals Unmethylated CpGs are often grouped in clusters called CpG islands, which are present in the 5′ regulatory regions of many genes. In many disease processes, such as cancer, gene promoter CpG islands acquire abnormal hypermethylation, which results in transcriptional silencing that can be inherited by daughter cells following cell division. Alterations of DNA methylation have been recognized as an important component of cancer development. Hypomethylation, in general, arises earlier and is linked to chromosomal instability and loss of imprinting, whereas hypermethylation is associated with promoters and can arise secondary to gene (oncogene suppressor) silencing, but might be a target for epigenetic therapy.

DNA methylation may affect the transcription of genes in two ways. First, the methylation of DNA itself may physically impede the binding of transcriptional proteins to the gene, and second, and likely more important, methylated DNA may be bound by proteins known as methyl-CpG-binding domain proteins (MBDs). MBD proteins then recruit additional proteins to the locus, such as histone deacetylases and other chromatin remodeling proteins that can modify histones, thereby forming compact, inactive chromatin, termed heterochromatin. This link between DNA methylation and chromatin structure is very important. In particular, loss of methyl-CpG-binding protein 2 (MeCP2) has been implicated in Rett syndrome; and methyl-CpG-binding domain protein 2 (MBD2) mediates the transcriptional silencing of hypermethylated genes in cancer

Methylated cytosines in vertebrates largely occur in small inverted repeats, so-called CpG dinucleotides, allowing methylation on the Cs of both strands, diagonally across from each other. This enables DNA methylation information to be copied in vivo during the DNA replication that precedes cell division, because the methylation on the parent strand is used to guide the reintroduction of DNA methylation on the daughter strand. This copying of DNA methylation is carried out by the enzyme DNA methyltransferase 1 (DNMT1), a “maintenance” DNA methyltransferase, at the replication fork, in a complex containing many proteins.

DNA methylation at the 5 position of cytosine has the specific effect of reducing gene expression and has been found in every vertebrate examined.

The DNA methyltransferase (DNA MTase) family of enzymes catalyze the transfer of a methyl group to DNA. DNA methylation serves a wide variety of biological functions. There are three categories of DMA methyltransferase enzymes m6A (those that generate N6-methyladenine), m4C (those that generate N4-methylcytosine) and m5C (those that generate C5-methylcytosine). Three active DNA methyltransferases have been identified in mammals. They are named DNMT1, DNMT3A, and DNMT3B.

DNMT1 is the most abundant DNA methyltransferase in mammalian cells, and considered to be the key maintenance methyltransferase in mammals. It predominantly methylates hemimethylated CpG di-nucleotides in the mammalian genome. This enzyme is 7-to 100-fold more active on hemimethylated DNA as compared with unmethylated substrate in vitro, but it is still more active at de novo methylation than other DNMTs. The recognition motif for the human enzyme involves only three of the bases in the CpG dinuclotide pair: a C on one strand and CpG on the other. This relaxed substrate specificity requirement allows it to methylate unusual structures like DNA slippage intermediates at de novo rates that equal its maintenance rate. Like other DNA cytosine-5 methyltransferases the human enzyme recognizes flipped out cytosines in double stranded DNA and operates by the nucleophilic attack mechanism. In human cancer cells DNMT1 is responsible for both de novo and maintenance methylation of tumor suppressor genes. The enzyme is about 1,620 amino acids long (SEQ ID NO:1). The first 1,100 amino acids constitute the regulatory domain of the enzyme, and the remaining residues constitute the catalytic domain. These are joined by Gly-Lys repeats. Both domains are required for the catalytic function of DNMT1.

An E3 ubiquitin ligase is a ligase enzyme that combines with a ubiquitin-containing E2 ubiquitin-conjugating enzyme, recognizes the target protein that is to be ubiquinated, and causes the attachment of ubiquitin to a lysine on the target protein via an isopeptide bond. E3 ubiquitin ligases are alsoinvolved in other cellular processes, such as DNA methylation. E3 ubiquitin ligases fall into specific groups called ubiquitin-ligase families including a RING (Really Interesting New Gene) domain binds the E2 conjugase and might be found to mediate enzymatic activity in the E2-E3 complex and a HECT domain, which is involved in the transfer of ubiquitin from the E2 to the substrate. In molecular biology, a RING finger domain is a protein structural domain of zinc finger type which contains a Cys3HisCys4 amino acid motif which binds two zinc cations. This protein domain contains from 40 to 60 amino acids. Many proteins containing a RING finger play a key role in the ubiquitination pathway. The HECT domain is a protein domain found in ubiquitin-protein ligases.

Examples of E3 ligases include E3A, mdm2, Anaphase-promoting complex (APC), UBR5 (EDD1), SOCS/BC-box/eloBC/CUL5/RING, LNXp80, CBX4, CBLL1, HACE1, HECTD1, HECTD2, HECTD3, HECW1, HECW2, HERC1, HERC2, HERC3, HERC4, HUWE1, ITCH, NEDD4, NEDD4L, Parkin, PPIL, PRPF19, PIAS1, PIAS2, PIAS3, PIAS4, RANBP2, RNF4, RBX1, SMURF1, SMURF2, STUB1, TOPORS, TRIP12, UBE3A, UBE3B, UBE3C, UBE4A, UBE4B, UBOX5, UBR5, UHRF1, WWP1and WWP2.

Ubiquitin-like, containing PHD and RING finger domains, 1, also known as UHRF1, is an E3 ubiquitin ligase. The protein binds to specific DNA sequences, and recruits a histone deacetylase to regulate gene expression. The protein recruits the main DNA methyltransferase gene, DNMT1, to regulate chromatin structure and gene expression. Its expression peaks at late G1 phase and continues during G2 and M phases of the cell cycle. It plays a major role in the G1/S transition by regulating topoisomerase II alpha and retinoblastoma gene expression, and functions in the p53-dependent DNA damage checkpoint. Multiple transcript variants encoding different isoforms have been found for this gene.

In various embodiments, the DNMT1 or UHRF1 may be a conjugate protein. For example, the invention may utilize a DNMT1 conjugate protein or a UHRF1 conjugate protein in which one or more domains of each protein may be utilized. Alternatively, a DNMT1-UHRF1 protein conjugate is envisioned.

The polymerase chain reaction (PCR) is a biochemical technology in molecular biology used to amplify a single or a few copies of a piece of DNA across several orders of magnitude, generating thousands to millions of copies of a particular DNA sequence. PCR is used to amplify a specific region of a DNA strand (the DNA target). Most PCR methods typically amplify DNA fragments of between 0.1 and 10 kilo base pairs (kb), although some techniques allow for amplification of fragments up to 40 kb in size. The amount of amplified product is determined by the available substrates in the reaction, which become limiting as the reaction progresses.

A basic PCR set up requires several components and reagents. These components include: a) aDNA template that contains the DNA region (target) to be amplified; b) at least two primers that are complementary to the 3′ (three prime) ends of each of the sense and anti-sense strand of the DNA target; a DNA polymerase; c) deoxynucleoside triphosphates (dNTPs,), d) a buffer solution, providing a suitable chemical environment for optimum activity and stability of the DNA polymerase and f) monovalent cation potassium ions. Optionally, divalent cations, magnesium or manganese ions may be used; generally Mg2+ is used, but Mn2+ can be utilized for PCR-mediated DNA mutagenesis, as higher Mn2+ concentration increases the error rate during DNA synthesis.

Typically, PCR reaction consists of a series of 20-40 repeated temperature changes, called cycles, with each cycle commonly consisting of 2-3 discrete temperature steps. The cycling is often preceded by a single temperature step at a high temperature (>90° C.), and followed by one hold at the end for final product extension or brief storage. The temperatures used and the length of time they are applied in each cycle depend on a variety of parameters. These include the enzyme used for DNA synthesis, the concentration of divalent ions and dNTPs in the reaction, and the melting temperature (Tm) of the primers. The basic steps of PCR include:

1. Initialization step: This step consists of heating the reaction to a temperature of 94-96° C. (or 98° C. if extremely thermostable polymerases are used), which is held for 1-9 minutes. It is only required for DNA polymerases that require heat activation by hot-start PCR.

2. Denaturation step: This step is the first regular cycling event and consists of heating the reaction to 94-98° C. for 20-30 seconds. It causes DNA melting of the DNA template by disrupting the hydrogen bonds between complementary bases, yielding single-stranded DNA molecules.

3. Annealing step: The reaction temperature is lowered to 50-65° C. for 20-40 seconds allowing annealing of the primers to the single-stranded DNA template. Typically the annealing temperature is about 3-5° C. below the Tm of the primers used. Stable DNA—DNA hydrogen bonds are only formed when the primer sequence very closely matches the template sequence. The polymerase binds to the primer-template hybrid and begins DNA formation.

4. Extension/elongation step: The temperature at this step depends on the DNA polymerase used; Taq polymerase has its optimum activity temperature at 75-80° C., and commonly a temperature of 72° C. is used with this enzyme. At this step the DNA polymerase synthesizes a new DNA strand complementary to the DNA template strand by adding dNTPs that are complementary to the template in 5′ to 3′ direction, condensing the 5′-phosphate group of the dNTPs with the 3′-hydroxyl group at the end of the nascent (extending) DNA strand. The extension time depends both on the DNA polymerase used and on the length of the DNA fragment to be amplified. As a rule-of-thumb, at its optimum temperature, the DNA polymerase will polymerize a thousand bases per minute. Under optimum conditions, i.e., if there are no limitations due to limiting substrates or reagents, at each extension step, the amount of DNA target is doubled, leading to exponential (geometric) amplification of the specific DNA fragment.

5. Final elongation: This single step is occasionally performed at a temperature of 70-74° C. for 5-15 minutes after the last PCR cycle to ensure that any remaining single-stranded DNA is fully extended.

6. Final hold: This step at 4-15° C. for an indefinite time may be employed for short-term storage of the reaction.

The basic PCR reaction has many variations including Allele-specific PCR, Assembly PCR or Polymerase Cycling Assembly (PCA), Asymmetric PCR, Dial-out PCRDigital PCR (dPCR), Helicase-dependent amplification, Hot start PCR, In silico PCR (digital PCR, virtual PCR, electronic PCR, e-PCR), Intersequence-specific PCR (ISSR), Inverse PCR, Ligation-mediated PCR, Methylation-specific PCR (MSP), Miniprimer PCR, Multiplex Ligation-dependent Probe Amplification (MLPA) Multiplex-PCR, Nanoparticle-Assisted PCR (nanoPCR), Nested PCR, Overlap-extension PCR or Splicing by overlap extension (SOEing), PAN-AC, quantitative PCR (qPCR), Reverse Transcription PCR (RT-PCR), Solid Phase PCR, Suicide PCR, Thermal asymmetric interlaced PCR (TAIL-PCR), Touchdown PCR (Step-down PCR) and Universal Fast Walking. The methods described herein can be used in conjunction with any type of PCR.

A second aspect of the present invention is directed to a methylation-preserving PCR method. Methods in accordance with this aspect of the invention will generally include the steps of denaturing parent DNA samples into single stranded DNAs; copying the parent DNA strands using primers and polymerase to make daughter strands; methylating the daughter strands with DNMT1 and UHRF1; and repeating the process for a desired number of cycles, wherein in each cycle, the methylated daughter stands are taken as new parent strands in the next cycle.

A third aspect of the present invention is directed to a method of detecting and analyzing DNA methylation patterns in a sample. In some embodiments, methods in accordance with this aspect of the invention will generally include performing a methylation-preserving DNA amplification process as described above, following by interrogating the amplified DNA product for methylation patterns. Interrogation of the amplified DNA product may be performed by any methods commonly known in the art. Such methods typically incorporate a bisulfite treatment reaction followed by some sort of PCR, hybridization, or sequencing.

A fourth aspect of the present invention is directed to a reagent kit for performing a DNA methylation preserving PCR reaction. Kits in accordance with this aspect of the invention will generally include reagents for copying methylated DNAs, wherein said reagents comprises DNMT1 and UHRF1; and instructions encoded on a permanent medium wherein said instructions comprises steps for performing methods according to the first, second, or third aspect of the invention as described above. In some preferred embodiments, the DNMT1 and UHRF1 are recombinant DNMT1 and UHRF 1 expressed in E. coli.

A fifth aspect of the present invention is directed to a cancer detection/diagnostic assay based on DNA methylation patterns corresponding to a cancer. Assays in accordance with this aspect of the invention will generally include the steps of obtaining a biological sample from a patient suspected of having a cancer; processing the sample and amplifying methylated DNAs in the sample by performing a methylation-preserving amplification reaction as described above (either according to the first aspect or the second aspect); interrogating the amplified DNAs for methylation patterns; comparing the methylation patterns to reference patterns; and determining a diagnosis based on the comparison, wherein if the methylation patterns in the sample deviates from the patterns in the references, a diagnosis of cancer is determined.

As used herein, DNA methylation associated disease or disorder is any disease or disorder in the DNA methylation pattern is different from a normal or reference DNA methylation pattern. An example of a DNA methylation associated disease or disorder is cancer.

As used herein, the terms “cancer” and “cancerous” refer to or describe the physiological condition in mammals in which a population of cells are characterized by unregulated cell growth. Examples of cancer include, but are not limited to, carcinoma, lymphoma, blastoma, sarcoma, leukemia, benign or malignant tumors. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, brain, hepatic carcinoma and various types of head and neck cancer, neurofibromatosis type I or II. Other examples of such cancers include those that are therapy resistant, refractory or metastatic.

“Metastasis” as used herein refers to the process by which a cancer spreads or transfers from the site of origin to other regions of the body with the development of a similar cancerous lesion at the new location. A “metastatic” or “metastasizing” cell is one that loses adhesive contacts with neighboring cells and migrates via the bloodstream or lymph from the primary site of disease to invade neighboring body structures.

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “subject suspected of having cancer” refers to a subject that presents one or more symptoms indicative of a cancer (e.g., a noticeable lump or mass) or is being screened for a cancer (e.g., during a routine physical). A subject suspected of having cancer can also have one or more risk factors. A subject suspected of having cancer has generally not been tested for cancer. However, a “subject suspected of having cancer” encompasses an individual who has received an initial diagnosis but for whom the stage of cancer is not known. The term further includes people who once had cancer (e.g., an individual in remission).

As used herein, the term “subject at risk for cancer” refers to a subject with one or more risk factors for developing a specific cancer. Risk factors include, but are not limited to, gender, age, genetic predisposition, environmental exposure, previous incidents of cancer, preexisting non-cancer diseases, and lifestyle.

As used herein, the term “characterizing cancer in a subject” refers to the identification of one or more properties of a cancer sample in a subject, including but not limited to, the presence of benign, pre-cancerous or cancerous tissue, the stage of the cancer, and the subject's prognosis. Cancers can be characterized by the identification of the expression of one or more cancer marker genes, including but not limited to, the cancer markers disclosed herein.

As used herein, “providing a diagnosis” or “diagnostic information” refers to any information that is useful in determining whether a patient has a disease or condition and/or in classifying the disease or condition into a phenotypic category or any category having significance with regards to the prognosis of or likely response to treatment (either treatment in general or any particular treatment) of the disease or condition. Similarly, diagnosis refers to providing any type of diagnostic information, including, but not limited to, whether a subject is likely to have a condition (such as a tumor), information related to the nature or classification of a tumor as for example a high risk tumor or a low risk tumor, information related to prognosis and/or information useful in selecting an appropriate treatment. Selection of treatment can include the choice of a particular chemotherapeutic agent or other treatment modality such as surgery or radiation or a choice about whether to withhold or deliver therapy.

As used herein, the terms “providing a prognosis”, “prognostic information”, or “predictive information” refer to providing information regarding the impact of the presence of cancer (e.g., as determined by the diagnostic methods of the present invention) on a subject's future health (e.g., expected morbidity or mortality, the likelihood of getting cancer, and the risk of metastasis).

As used herein, the term “post surgical tumor tissue” refers to cancerous tissue (e.g., biopsy tissue) that has been removed from a subject (e.g., during surgery).

As used herein, the term “subject diagnosed with a cancer” refers to a subject who has been tested and found to have cancerous cells. The cancer can be diagnosed using any suitable method, including but not limited to, biopsy, x-ray, blood test, and the diagnostic methods of the present invention.

As used herein, the terms “biopsy tissue”, “patient sample”, “tumor sample”, and “cancer sample” refer to a sample of cells, tissue or fluid that is removed from a subject for the purpose of determining if the sample contains cancerous tissue, including cancer cells or for determining gene expression profile of that cancerous tissue. In some embodiment, biopsy tissue or fluid is obtained because a subject is suspected of having cancer. The biopsy tissue or fluid is then examined for the presence or absence of cancer, cancer cells, and/or cancer cell gene signature expression.

As described above, preferred embodiments for performing methylation-preserving amplification of DNAs will generally include the following steps:

STEP 1: Denature+copy DNA 1× using primers and polymerase (regular or heat-stable; akin to 1 cycle of PCR);

STEP 2: Methylate the daughter strand with DNMT and HRF1. Repeat both steps for several cycles.

To analyze the methylation pattern, the amplified DNA products may then be subjected to various interrogation methods. Interrogation of the DNA methylation information of the products may be done by using any of the standard technologies, most of which incorporate a bisulfite treatment reaction followed by some sort of PCR, hybridization, or sequencing.

The above described process will thus allow the analysis of DNA methylation information in very small samples. Because normal PCR erases DNA methylation information, the current approach involves a chemical treatment (bisulfite conversion) that embeds DNA methylation information into the DNA sequence by deaminating all unmethylated cytosines (turning them into Us, which are subsequently converted to Ts during PCR). Methylated cytosines are protected from deamination and are thus preserved as Cs. When Cs are later detected, their positions will be assumed to have been methylated. A major problem is that bisulfite conversion is very damaging to the DNA and can destroy up to 90% of it. When small samples are used, this greatly diminishes the sensitivity of DNA methylation detection.

The approach described herein can be used to amplify DNA before bisulfite conversion. Because the DNA methylation patterns can be maintained, this pre-amplification increases the sensitivity of DNA methylation detection. This will be of value for use of methylated DNA as biomarkers in bodily fluids, and any other approach in which very small quantities of DNA are available, such as single cell approaches that examine DNA methylation information.

The various aspects of the present invention will have a number of advantages over prior art methods of DNA methylation detection and analysis. As explained above, DNMT1 by itself is quite promiscuous, introducing “new” methylgroups into unmethylated CpG dinucleotides. This is a great problem. In some preferred exemplary embodiments, the accuracy of maintaining DNA methylation information by DNMT1 and UH RF1 was over 98%. In three replication cycles, this would maintain 94% of DNA methylation. It is important to note that DNMT1 does not need UHRF1 for this. (We have determined that DNMT1 alone can also copy the information well). However, the accuracy of DNMT1 and UH RF1 in leaving unmethylated CpGs unmethylated was 100%. This contrasts dramatically with using DNMT1 alone, which introduces “new” DNA methylation at 29% of sites, thus showing and accuracy of only 71% with respect to not methylating sites that are unmethylated. In three replication cycles, this could result in only 35% of unmethylated sites remaining unmethylated.

The following examples are provided to further illustrate the embodiments of the present invention, but are not intended to limit the scope of the invention. While they are typical of those that might be used, other procedures, methodologies, or techniques known to those skilled in the art may alternatively be used.

### EXAMPLE 1

### Methylation Preservation

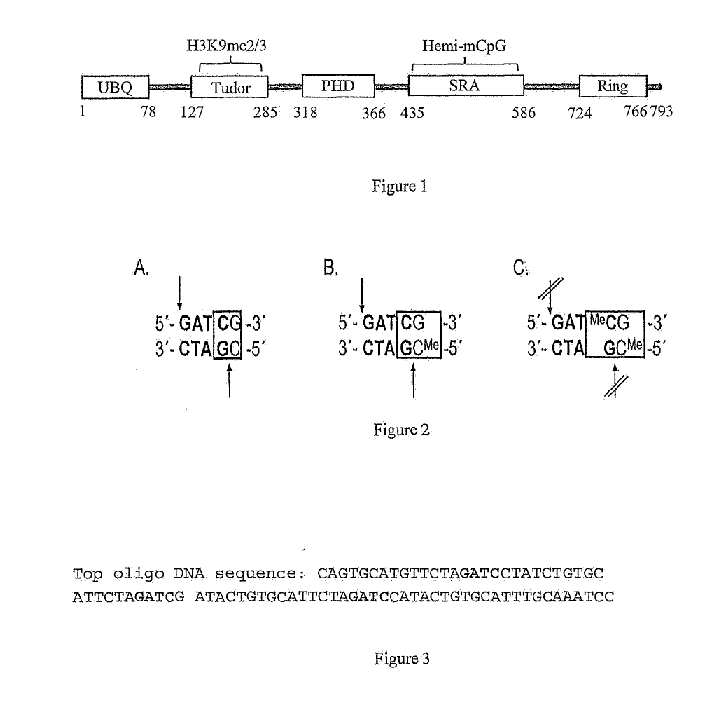

Cloning & Protein Expression and Purification. The full length human DNMT1 cDNA, encoding a 1632 amino acid protein was generously donated by Dr. Art Riggs and was cloned into a pET vector such that the resulting recombinant protein would have a (His)₆-tag at the C-terminus. The plasmid was transformed into E. coli BL21 containing a second compatible plasmid that provides a repressor and additional copies of rare codons. Protein production was induced using 0.5 mM IPTG at 16° C. for 16 hours. Cells were pelleted and lysed by sonication in the sonication buffer (10 mM Tris pH7.4, 150 mM NaCl, and 0.5% Trixton-100) using 550 Sonic Dismembrator (Fisher Scientific). The cell lysate was separated to supernatant and pellet by centrifugatiom at 13000 rpm, and DNMT1 protein was purified using 500 uL Qiagen Ni-NTA agarose beads mixed in the supernatant. The protein was eluted from the beads by adding 100 mM imidazole in the sonication buffer. The protein was dialyzed in the reaction buffer (30 mM Tris pH 7.5, 150 mM NaCl, 0.5% Trixton-100, and 40% glycerol) at 4° C. for 12 hours and stored at −80° C. The full-length human UHRF1 cDNA (from the NIH-funded Mammalian Genome Collection) and several deletion mutants constructed by us using PCR were similarly cloned in a pET vector and expressed at 26° C. for 8 hours and purified using a similar method as for DNMT1.UHRF1⁴¹⁵⁻⁶²⁰ includes the SRA domain, and UHRF1⁴¹⁵⁻⁷⁹³ contains the SRA domain and the whole C-terminal region of UHRF1 (FIG. 1). All plasmids were confirmed by sequencing.

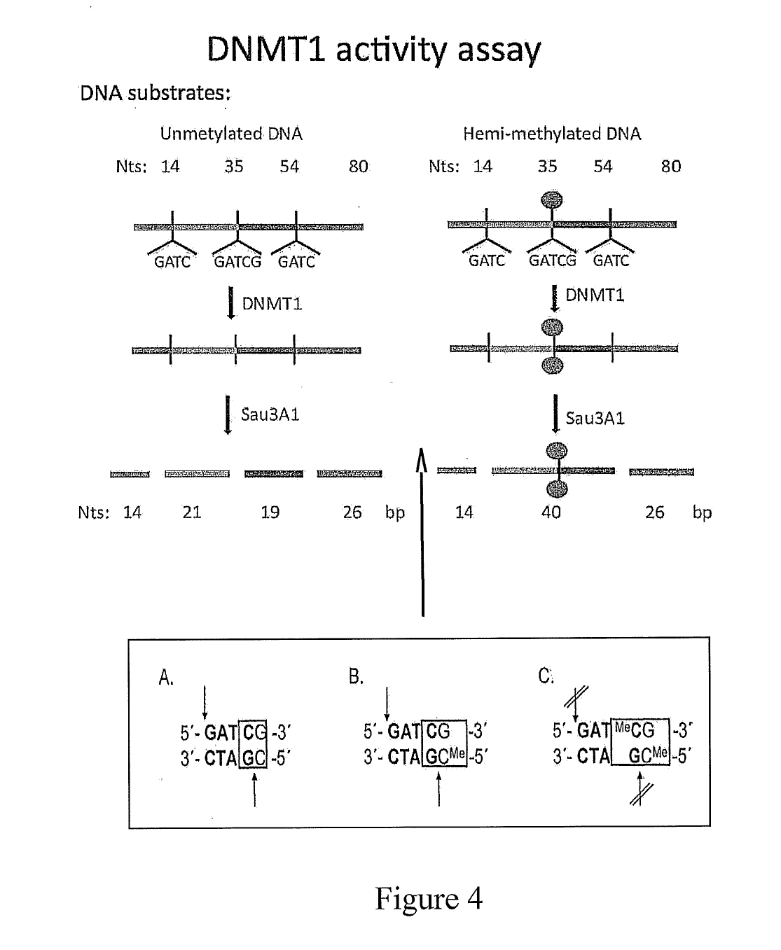

DNMT1 Activity Assay on a Single Methylated CpG Site. To test the activity of our recombinant DNMT1, we developed a strategy using the restriction enzyme Sau3AI. Sau3AI can digest the GATC restriction site, but cleavage is blocked by some forms of overlapping CpG methylation. A synthetic 80-nt DNA fragment was designed to contain three GATC Sau3A1 sites, the central of which (GATCG) abuts a CpG dinucleotide (FIG. 2). Single-stranded DNA that is unmethylated or methylated at the relevant Cs can be annealed to provide unmethylated, hemi-methylated or fully methylated substrates at this CpG. If the lower strand is methylated but the top one is not (hemi-methylated DNA), Sau3A1 can still digest the restriction site because the methylated cytosine lies outside the GATC sequence. However, if the top strand is methylated, the methylated cytosine lies in the restriction site and blocks digestion. Thus, the 80-nt DNA fragment has a central GATCG that can be methylated on the top or bottom strand and two flanking GATC sites that are never methylated and function as Sau3AI digestion controls (see FIGS. 3-5). This allows de novo and maintenance DNA methyltransferase activity to be detected. De novo DNA methylation activity has been reported in the literature, including by Goyal et al. (2006) 34(4): 1182-88 (the entire content of which is incorporated herein by reference).

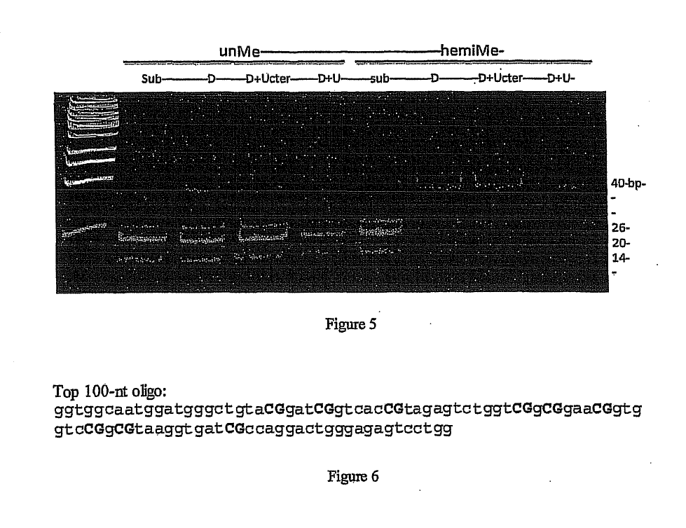

300 ng double-stranded target DNA that was un- or hemi-methylated (on the bottom strand) (300 ng) was mixed with 500 nM DNMT1 and 160 uM S-adenosyl methionine (SAM) in methylation buffer (30 mM Tris pH7.5, 100 ug/mL BSA, and 1 mM EDTA) at 37° C. degree for 16 hours to allow exhaustive methylation. To examine whether UHRF1 could inhibit inaccurate de novo DNA methylation activity of DNMT1 (i.e. methylation of an unmethylated CpG site), we added 500 nM of the full-length UHRF1 protein or deletion mutants into the methylation reactions. Following methylation incubation, the DNA fragments were purified using a kit (Qiagen, PCR purification kit and cat #28104) and digested with Sau3A1 (NEB) at 37° C. for one hour. The result was examined by running the samples on a 20% TBE acrylamide gel (FIG. 5).

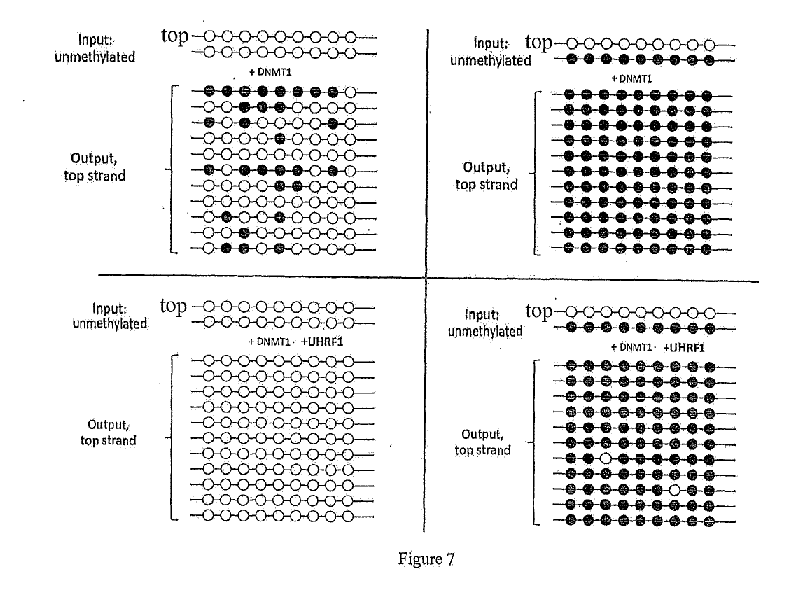

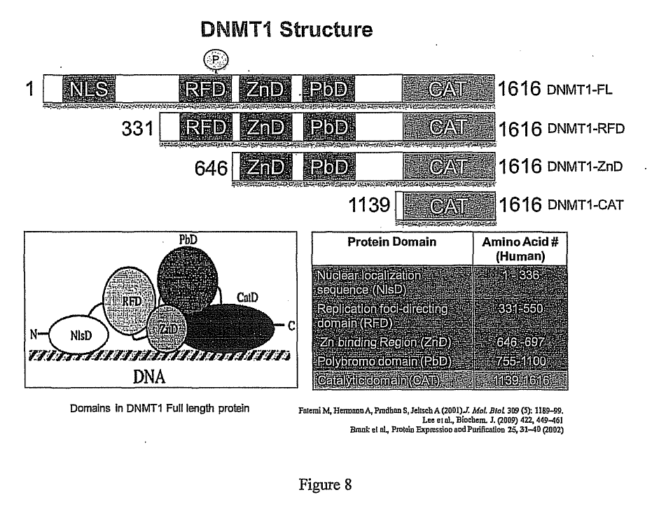

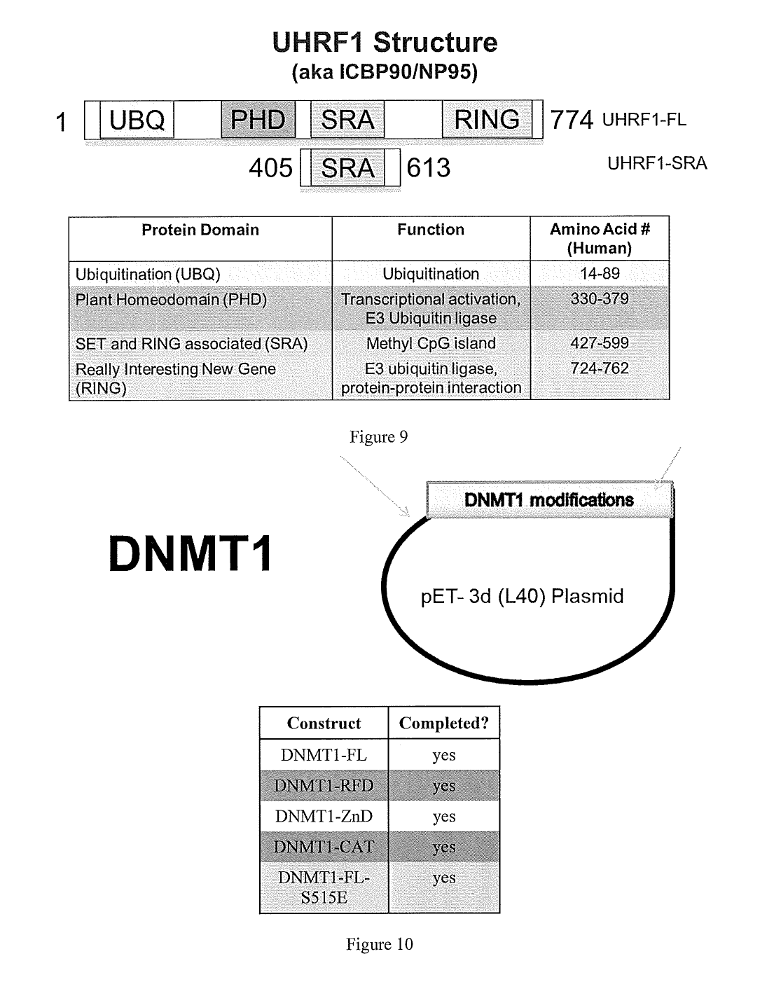

DNMT1 Methylation Activity on a Substrate with Multiple Un- or Hemi-Methylated CpG Sites. To examine DNMT1 methylation activity on a substrate with multiple un- or methylated CpG sites, a 100-nt DNA fragment (FIG. 6) (300 ng) containing nine un- or hemi-methylated (on the bottom strand) CpG sites was mixed with 500 nM DNMT1 and 160 uM SAM in the methylation buffer at 37° C. degree for 16 hours to allow exhaustive methylation. The DNA fragment was bisulfite-treated using the EZ DNA Methylation kit (Zymo research). The bisulfite-treated DNA was amplified by PCR using Taq polymerase (Invitrogen, cat #10342-020) for 3 cycles (M13 forward primer: GTAAAACGACGGCCA (SEQ ID NO: 5) and M13 reverse primer: CAGGAAACAGCTATGAC (SEQ ID NO: 6) and cloned into TA cloning vector (TOPO TA cloning kit, Invitrogen). At least 10 cloned samples for each test were sequenced for the top strand (GENGWIZ; SEQ ID NO: 7), to determine which CpGs became methylated (FIG. 7). Thus far, 500 nM full-length UHRF1 has been tested and it shows full inhibition of de novo DNMT1 methylation activity (FIG. 7).

### EXAMPLE 2

### Lung Cancer

Lung cancer is the leading cause of cancer death in the United States for both men and women. Aberrant DNA patterns have been observed in variety of cancers, including lung cancer. The presence of differentially methylated DNA elements originating from tumors poses a unique opportunity for early detection, as the aberrantly methylated DNA is shed into the blood stream and could, in theory be detected. However, sensitivity remains a problem due to the minute amount of DNA being shed.

A method is envisioned that would simultaneously copy and amplify the epigenetic DNA methylation signature associated with that DNA molecule. This may be achieved by utilization of DNA methyl-transferase, DNMT1, in combination with it's DNA targeting partner, UHRF1. This will allow for detection of aberrant DNA methylation patterns from trace amount of material found in blood, and therefore could be implemented as a method of early cancer detection, which could be combined with CT screening.

Objectives

Develop a method for methylation-preserving FCR using full length DNMT1 or fragments thereof in combination with full length UHRF1 or fragments thereof as individual or fusion proteins.

Identify the most efficient enzyme combination above and determine the efficacy of methylation-preserving FCR on methylated DNA with different density CpGs as well as different levels of methylation.

Determine efficacy of methylation-preserving PCR to detect DNA methylation using plasma from unidentified lung cancer patients and non-cancer controls.

Results

Recombinant DNMT1 fragments were cloned into bacterial expression vectors and successfully expressed. SRA fragment was successfully produced and purified from bacterial cells. Methylation assay detected DNMT1 activity, but may have non-specific effects SRA domain bound more specifically to hemi-methylated DNA than UHRF1, DNMT1 did not bind to methylated DNA

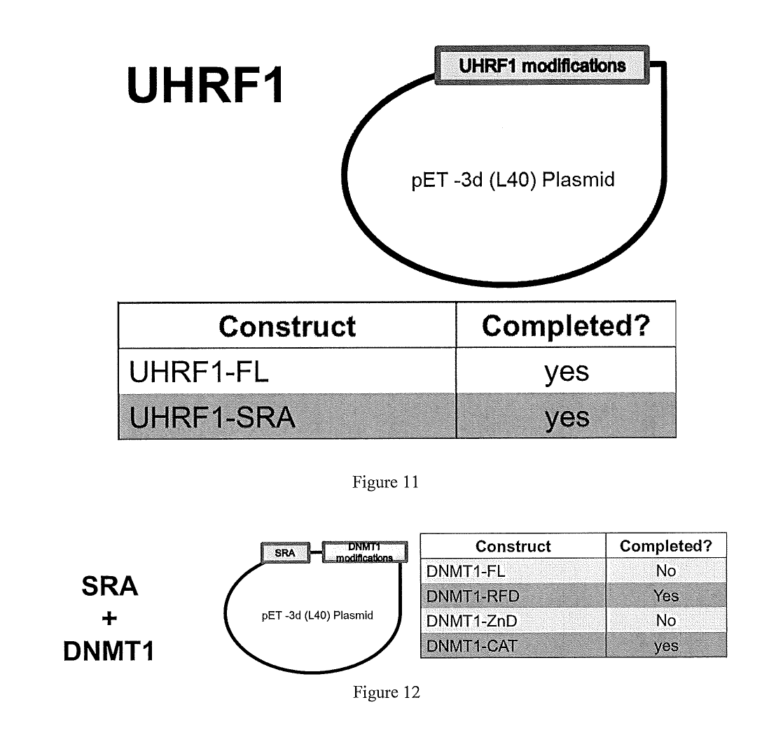

DNMT1 and SRA Domain Cloning (FIGS. 10-12). Full length DNMT1 (DNMT1-FL) and truncations were cloned into the pET-3d (L40) bacterial expression plasmid for recombinant protein production. SRA domain, originally subcloned from UHRF1, was transplanted from pXC666 into the pET bacterial expression plasmid for recombinant protein production.

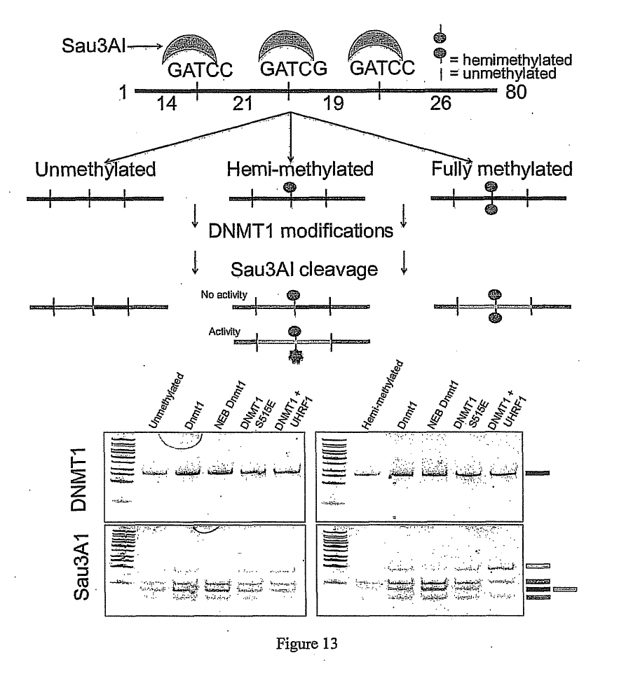

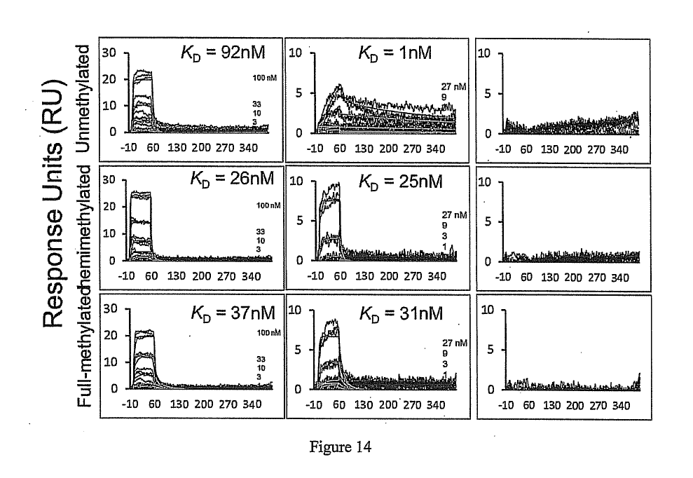

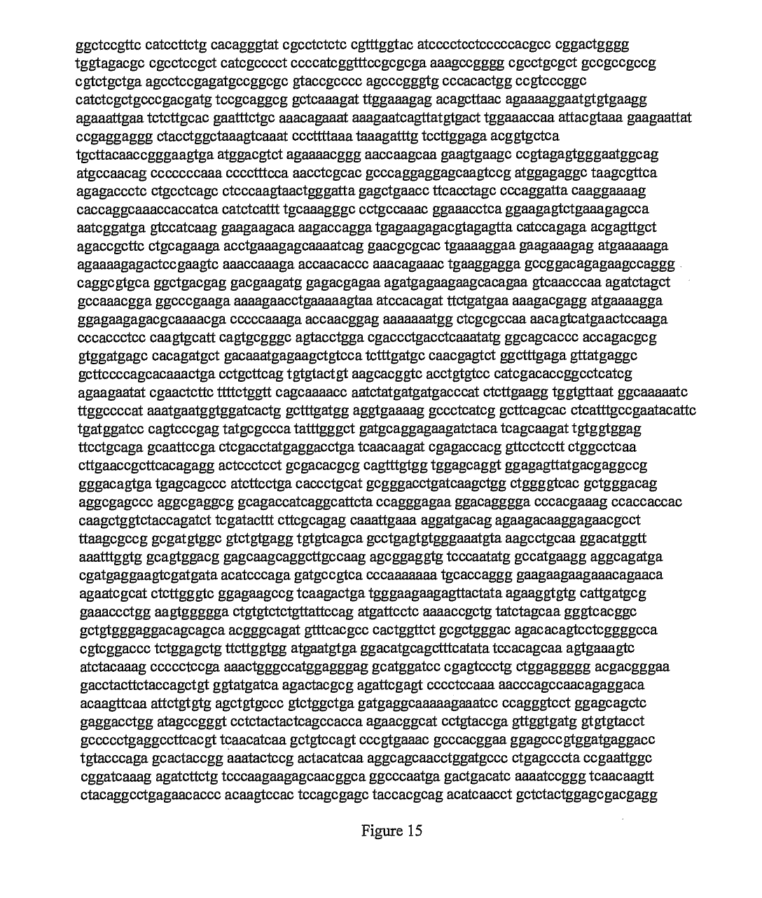

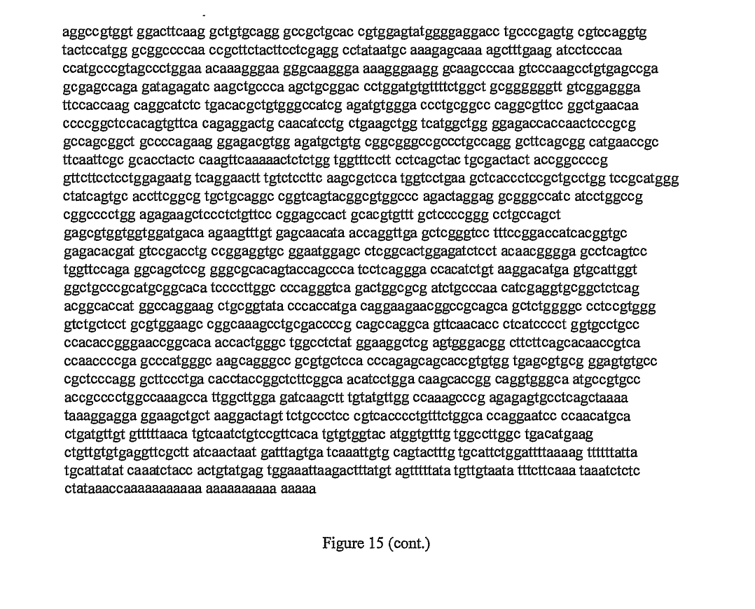

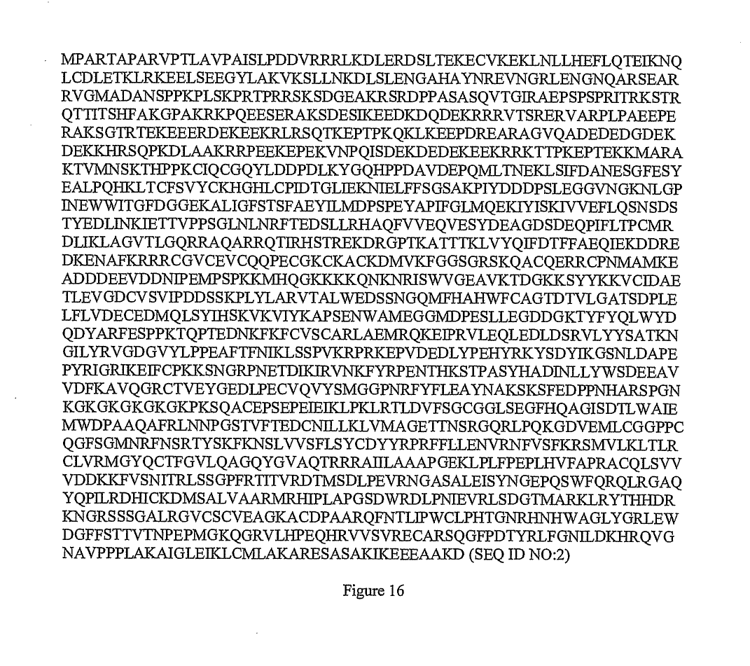

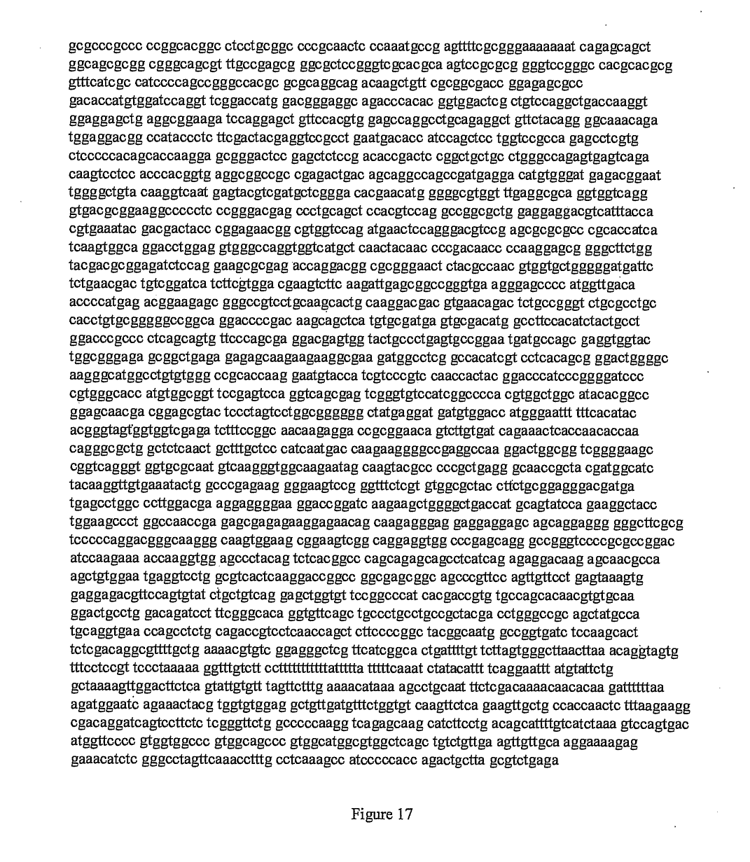

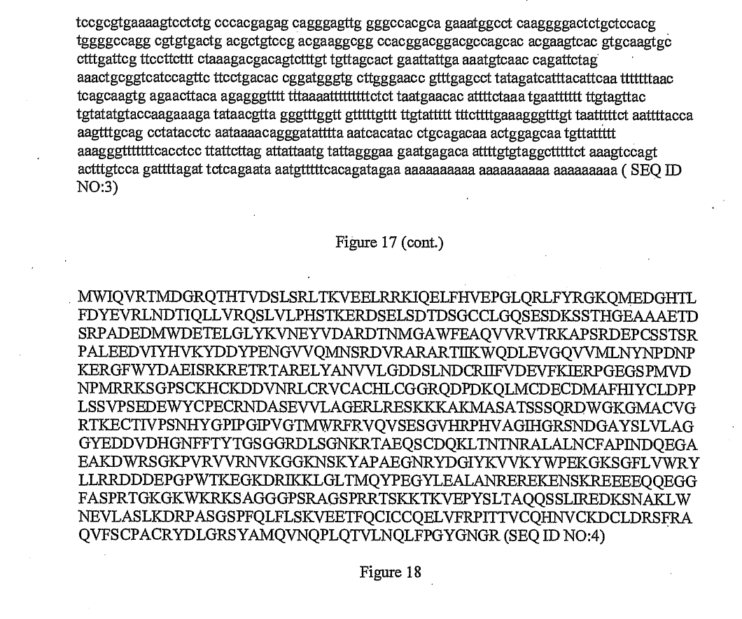

Results of DNMT1 methylation assay is shown in FIG. 13, while FIG. 14 is a series of graphical illustrations showing kinetic activity.

Although the present invention has been described in terms of specific exemplary embodiments and examples, it will be appreciated that the embodiments disclosed herein are for illustrative purposes only and various modifications and alterations might be made by those skilled in the art without departing from the spirit and scope of the invention as set forth in the following claims.

Additional details are further provided in the attachments in the Appendix section.

All references disclosed herein are incorporated herein by reference in their entirety.

What is claimed is:

1. A method of copying a methylated nucleic acid molecule comprising:
a) copying a first nucleic acid molecule into a plurality of nucleic acid molecules; and
b) contacting the plurality of nucleic acid molecules with a DNA methyltransferase enzyme and an E3 ubiquitin ligase, thereby copying the methylated nucleic acid molecule.

2. The method of claim 1, wherein the plurality of nucleic acids comprises at least 90% of the methylation pattern of the first nucleic acid.

3. The method of claim 1, wherein the first nucleic acid is double stranded or single stranded.

4. The method of claim 3, wherein the double stranded nucleic acid is denatured.

5. The method of claim 1, wherein the first nucleic acid is contacted with at least one nucleic acid primer and a DNA polymerase.

6. The method of claim 1, wherein step (a) further comprises annealing at least one nucleic acid primers to the first nucleic acid molecule.

7. The method of claim 6, wherein step (a) further comprises annealing two or more nucleic acid primers to the first nucleic acid molecule.

8. The method of claim 7, further comprising amplifying the first nucleic acid molecule with the DNA polymerase.

9. The method of claim 1, wherein the DNA methyltransferase is DNA (cytosine-5-)-methyltransferase 1 (DNMT1) or a functional fragment thereof.

10. The method of claim 1, wherein the E3 ubiquitin ligase is ubiquitin-like with PHD and ring finger domains 1 (UHRF1) or a functional fragment thereof.

11. The method of claim 1, further comprising analyzing the methylation pattern of the plurality of nucleic acid molecules.

12. The method of claim 11, wherein the methylation pattern is determined by the method selected from the group consisting of PCR, sequencing, restriction digestion, hybridization, bisulfate treatment or a combination thereof.

13. The method of claim 1, wherein steps (a) and (b) are repeated for a plurality of cycles.

14. The method of claim 13, wherein the plurality of nucleic acid molecules serve as the first nucleic acid molecule in each subsequent cycle.

15. A method preserving a methylation pattern in a nucleic acid amplification assay comprising:
a) copying a first nucleic acid molecule having a methylation pattern into a plurality of nucleic acid molecules; and
b) methylating the plurality of nucleic acid molecules by contacting the plurality of nucleic acid molecules with a DNA methyltransferase enzyme and an E3 ubiquitin ligase, wherein the methylation pattern of the first nucleic acid molecule is preserved in the plurality of nucleic acid molecules, thereby preserving a methylation pattern in the nucleic acid amplification assay.

16. The method of claim 15, wherein the plurality of nucleic acids comprises at least 90% of the methylation pattern of the first nucleic acid.

17. The method of claim 15, wherein the first nucleic acid is double stranded or single stranded.

18. The method of claim 17, wherein the double stranded nucleic acid is denatured.

19. The method of claim 15, wherein the first nucleic acid is contacted with at least one nucleic acid primer and a DNA polymerase.

20. The method of claim 19, wherein the first nucleic acid is contacted with at least two nucleic acid primers.

21-53. (canceled)
